(19) United States
(12) Patent Application Publication
(10) Pub. No.: US 2005/0279366 A1
(43) Pub. Date: Dec. 22, 2005

(54) Surgical drape system with catheter pouch

(76) Inventor: Adrienne Adragna, North Canton, OH (US)

Correspondence Address: KIMBERLY-CLARK WORLDWIDE, INC., 401 NORTH LAKE STREET, NEENAH, WI, 54956, US

(21) Appl. No.: 10/872,908

(22) Filed: Jun. 21, 2004

(51) Int. Cl. A61F 5/37; A61B 19/00

(52) U.S. Cl. 128/849

(57) ABSTRACT

A surgical drape system is provided including a drape component and a pouch component. The drape component has a main sheet portion and may have a fenestration for providing access to an operative site on a patient. The pouch component may be created from a rear sheet and a front sheet superimposed one over the other so as to create an envelope having an open top, sealed sides, and a sealed bottom. The open top is fastened proximal to the sealed bottom so that a first portion of a first surface is juxtaposed with a second portion of the first surface. This configuration results in an overlapping of the envelope to form a folded pouch which is adapted to contain a catheter therein.

### BACKGROUND OF THE INVENTION

The present invention relates generally to surgical drapes, and more particularly to surgical drapes adapted to work in conjunction with pouches, especially those surgical drapes for use in procedures that include the insertion of a catheter into a vein or artery of a patient.

Drapes are used during surgical procedures, in part, to provide a sterile field about the surgical site and during other treatment procedures requiring the maintenance of a sterile environment. When used during surgery, drapes prevent blood and other bodily fluids from cross contaminating the sterile field. There are a variety of different surgical drapes that are available. Although some surgical drapes are designed for specific surgical procedures, certain surgical procedures still require surgical personnel to modify or adapt available surgical drapes in an attempt to create a viable drape.

Cardiac catheterization and angiography procedures are surgical procedures that, due to their nature, present special requirements for a surgical drape. Cardiac catheterization is the introduction of a catheter (a long slender tube) into the heart in order to obtain information about the structure and function of the heart, the cardiac valves, the coronary arteries, and the like. The catheter is typically introduced through an artery or vein, such as the femoral or brachial artery. Typically, the catheter is introduced through the artery to determine the pressure within the heart, determine abnormalities in the structure of the heart, and the like. During the surgical procedure, the catheter is manipulated by the surgeon through the artery into the heart. Problems may be encountered with typical catheterization procedures due to the fact that catheters must be inserted within the artery, maintained in position, and manipulated by the surgeon during the procedure, all while sterility is maintained.

There is therefore a need for an improved means which would better assist the surgeon in performing the catheterization procedure, while assisting the surgeon in maintaining sterility.

### SUMMARY OF THE INVENTION

The present invention is drawn to a surgical drape system having a drape component and a pouch component. The drape component has a main sheet portion for covering at least a substantial portion of a patient. The drape component may have a fenestration for providing access to an operative site on a patient. The pouch component may be created from a rear sheet and a front sheet superimposed one over the other so as to create an envelope having an open top, sealed sides, and a sealed bottom. The open top would then be fastened proximal to the sealed bottom so that a first portion of a first surface is juxtaposed with a second portion of the first surface. This configuration would result in an overlapping of the envelope so as to form a folded pouch. The folded pouch would be adapted to contain a catheter therein. A fastener may be affixed to the second sheet for securing the pouch to a surface.

In some embodiments, the pouch may include sealed portions that are liquid tight. The open top may contain a closure and that closure may also be made liquid tight. The pouch fastener may be removably securable to the surface of the drape by means of a hook and loop fastener, tape, or other means.

In another embodiment, the present invention is drawn to a surgical drape system for use with a catheterization procedure that includes a drape having a main sheet for covering at least a substantial portion of a patient and a fenestration through the main sheet for access to a catheterization site. The system also includes a pouch located proximal to the catheterization site for containing a catheter for use in the procedure. The pouch has a first surface and a second surface secured to one another so as to form a top edge and a bottom edge joined by two opposing side edges. The side edges and bottom edge are sealed closed. The top edge is fastened proximal to the bottom edge so that a first portion of the first surface is juxtaposed with a second portion of the first surface thereby overlapping the pouch into a folded orientation. Such a system may contemplate that the first and second portions of the first surface are removably fastenable to one another so as to enable placement and control of medical lines and tubes between the first and second portions. The pouch itself may also be removably fastenable to the surgical drape.

In some embodiments, the pouch may be adapted to collect fluid run-off. Moreover, the pouch may be adapted to collect and seal within, for disposal; the catheter, medical waste, and fluid run-off resulting from the catheterization procedure while enabling the drape to remain in place for performance of subsequent procedures.

In yet another embodiment, a method of manufacturing a pouch is disclosed.

Such a method includes providing a sheet material having a plurality of edges. Two of the edges comprise a first and a second edge disposed opposite one another so as to define a length from the first edge to the second edge. The sheet material is overlapped so that the first and second edges are placed proximal to one another. The overlapping sheet material is sealed to itself to define a third edge and a fourth edge disposed opposite one another. This creates an envelope having an open top defined by the first and second edges, sealed sides defined by the third and fourth edges, and a sealed bottom. Next the envelope is overlapped so that the sealed bottom is placed proximal to the open top where the sealed bottom is fastened proximal to the open top thereby forming a pouch folded upon itself.

Such a surgical drape system may be found useful in surgical procedures where it is desirable to perform any or all of; collection of excess fluids that may escape from a surgical site, provide a means to secure surgical tubes and lines in specific positions throughout the surgical procedure, as well as provide a means to store surgical instruments and/or materials to be used in the surgical procedure.

These and other objects are achieved by the surgical drape system disclosed and claimed herein.

### BRIEF DESCRIPTION OF THE DRAWINGS

FIG. 1 is a diagrammatic view of an exemplary surgical drape system in use on a patient in accordance with the present invention.

FIG. 2 is a diagrammatic view of an exemplary construction of a pouch for use on the surgical drape system of FIG. 1.

FIGS. 3 and 4 are diagrammatic views of subsequent steps in the construction of the pouch for use on the surgical drape system of FIG. 1.

FIG. 5 is a diagrammatic view of one pouch embodiment for use on the surgical drape system of FIG. 1.

FIG. 6 is a diagrammatic view of an alternative pouch embodiment for use on the surgical drape system of FIG. 1.

FIG. 7 is an enlarged view of the seal of the FIG. 5 pouch.

### DETAILED DESCRIPTION OF THE INVENTION

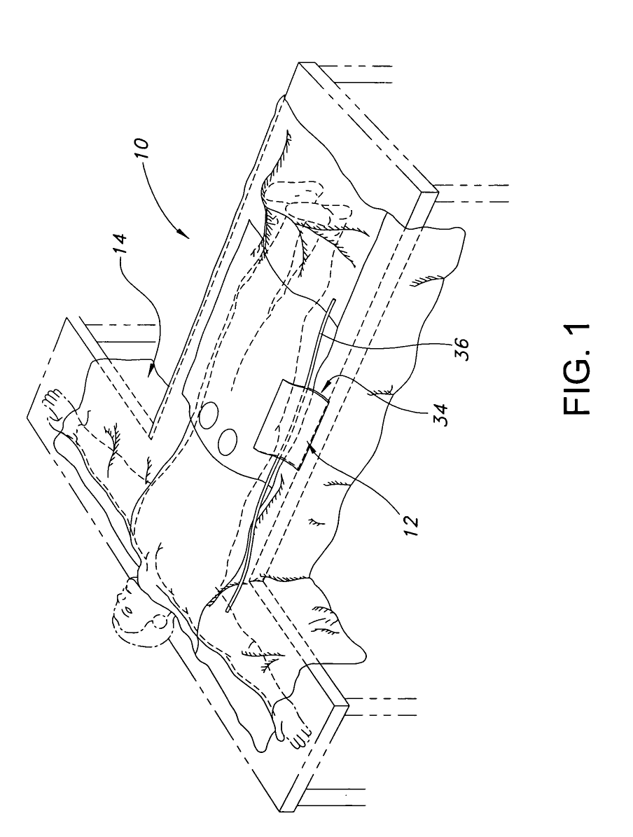

Referring to FIG. 1, the present invention is directed to an improved surgical drape system 10 that includes a catheter pouch 12 adapted to be used with a drape 14. The pouch 12 may be manufactured and sold alone or affixed to a drape 14 selected so as to be appropriate to the type of surgery or catheterization procedure to be performed. The pouch 12 would be designed to contain a catheter 16 (shown in FIG. 5) therein but may be made to serve additional purposes as well.

For example, a pouch in accordance with the present invention may be used to collect excess fluids that may escape from the surgical site thereby minimizing contamination of the operating room by the prevention of fluids from dripping onto the floor. Moreover, the collection of patient bodily fluids, including blood, would also be beneficial to operating personnel in that it may serve to minimize the potential transfer of blood-borne pathogens.

The pouch may also be adapted to serve as a means to secure surgical tubes and lines 36 in various configurations as depicted in FIG. 1 so that they remain in a desirable position throughout the surgical procedure that is to be performed. Another potential use for the pouch would be for the storage of surgical instruments and/or materials including waste materials such as gauze that may be used or generated during the surgical procedure. The usefulness and flexibility of such a pouch may be enhanced by enabling the pouch to be movable during the procedure as well as disposable at such time that the procedure has been completed. Such a pouch may be useful for any one individual or any combination of these purposes.

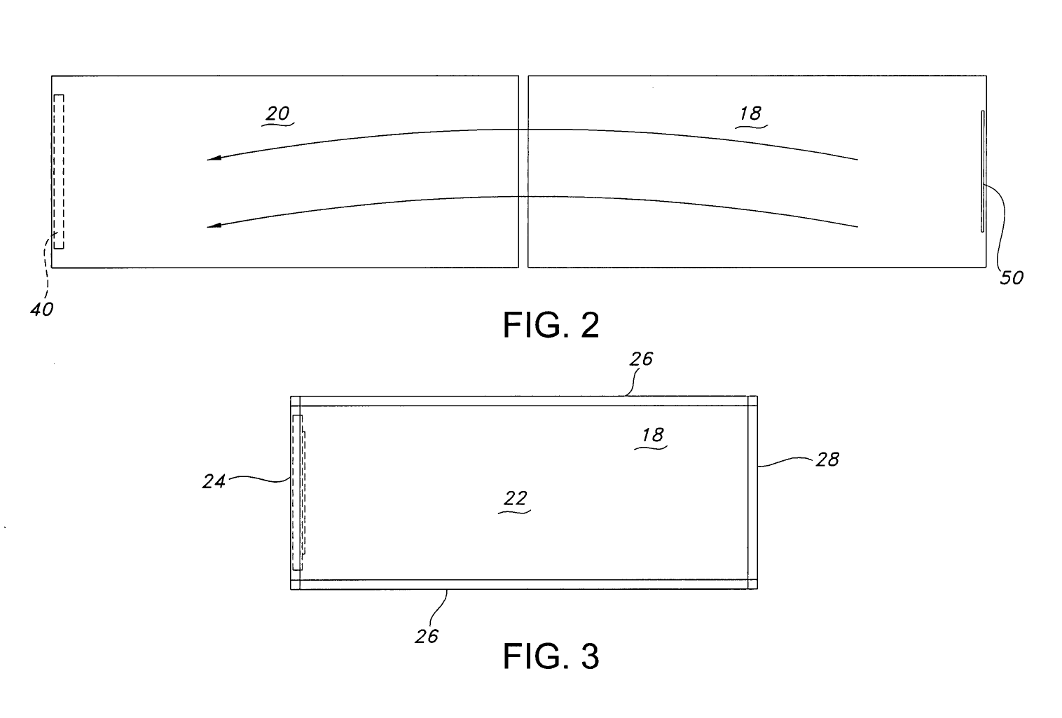

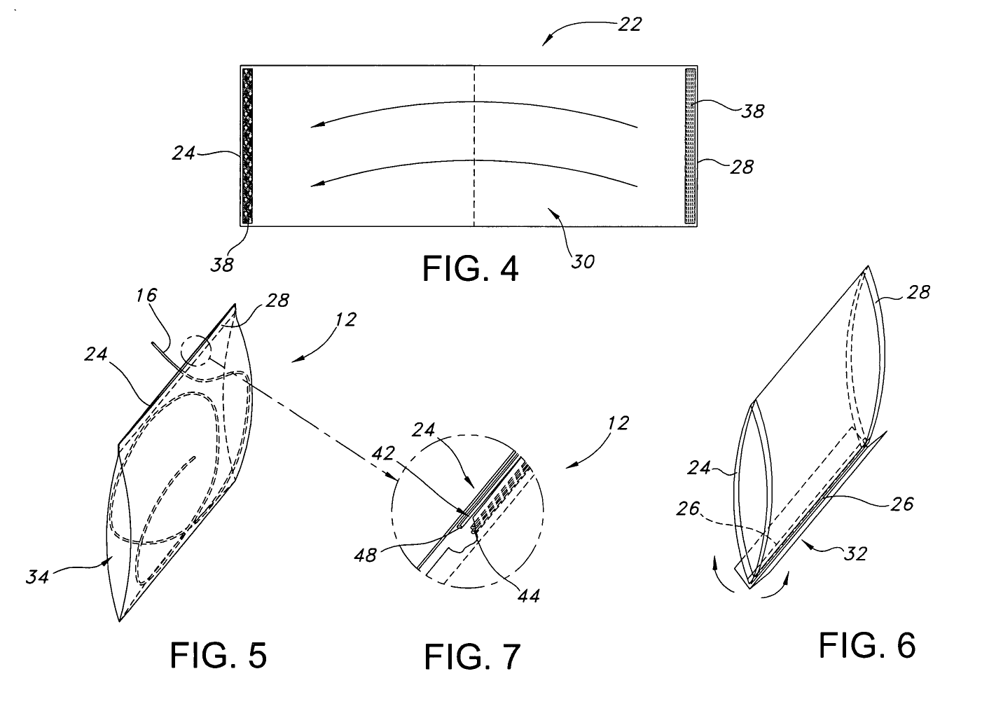

Looking now to FIGS. 2 and 3, it can be seen that the pouch 12 may be constructed as a first sheet 18 superimposed and sealed to a second sheet 20 so as to initially create an envelope 22 having an open top 24, sealed sides 26, and a sealed bottom 28 as shown on FIG. 3. As depicted in FIG. 4, the open top 24 is subsequently folded over and fastened proximal to the sealed bottom 28 so that a surface 30 is overlapped and a portion of that surface 30 is juxtaposed with another portion of the surface 30. In this way, the envelope 22 is formed into a loosely folded or overlapped pouch 12 as shown on FIG. 5.

Alternatively, a single sheet, such as first sheet 18 may be folded once over, the sides 26 sealed, and the bottom made liquid tight due to the nature of the fold itself. This is identical to the procedure described above in creating the pouch from the envelope and depicted in FIGS. 4 and 5, and therefore this is not separately illustrated. As such, the remainder of the pouch may be constructed similar to that described above, that is, it is folded over once more and the top 24 secured to the bottom 28.

In any of the embodiments, adhesive may be used to seal those edges that are meant to be sealed, although thermal, chemical, and/or mechanical bonding may also be used to form the sealed edges. Other methods of sealing the edges 26 are contemplated as well. One such example shown in FIG. 6, provides strips of material 32 that overlap the superposed edges of the envelope, for example, on an exterior portion of the envelope. The strips 32 are sealed to the envelope thus encasing and sealing the sides 26 to one another.

Looking back to FIG. 5, in some embodiments, the overlapped sides 26 of the pouch 12 are not sealed to one another though they would be sealed in the previous step thereby forming the envelope 22. This configuration provides a path or passage 34 as shown on FIGS. 1 and 5, through which surgical tubes and/or lines 36, as shown on FIG. 1 may be made to pass. The tubes and lines are thus provided with a means to locate, run, and contain them so that they do not interfere with the procedure that is to be performed. Of course, there is no requirement that the edges of the pouch be completely open. As shown in the FIG. 6 cross-section, the side 26 may be completely sealed. It may be sealed in steps, that is, the side may first be sealed when the envelope 22 is formed and subsequently sealed yet again to form the pouch 12. Alternatively, the sealing of the four layers shown may be done in a single step upon formation of the pouch.

To make it easier to access the passage 34, a fastener or fasteners 38 may be provided to secure the open top 24 to the sealed bottom 28. The fastener 38, shown in FIG. 4, may be constructed so that it enables limited use such as would occur with a tape or adhesive or it may be designed to be reusable such as would occur with a hook and loop type fastener or reusable snaps.

The sheets 18 and/or 20 may be constructed so as to be opaque, translucent, or transparent in whole or in part. The materials for constructing the pouch are well known and understood by those skilled in the art. Some suitable materials for manufacturing the pouch include but are not limited to films and nonwoven materials. Since the pouch 12 is adapted to contain a catheter 16 therein, it may be desirable for the outer sheet of the pouch 12 to be transparent or otherwise enable visibility of the catheter while it is contained within the pouch. As such a transparent film would be useful for this purpose.

As stated, the pouch 12 may be a stand-alone component that is attached to the drape 14 just prior to the surgical procedure when the patient is being prepped. This would enable optimal placement of the pouch and repositioning of the pouch as desired by the surgical team. As such, some manner or means would desirably be provided to secure the pouch 12 to the drape 14. Various possibilities exist to perform this function including adhesive, tape, or some other semi-permanent method. Alternatively a fastener 40 shown on FIG. 2 may be provided. In one embodiment the fastener 40 may comprise a hook and loop type fastener. Of course other fasteners such as clips and/or snaps may be used. Reusable fasteners such as hook and loop type fasteners may be utilized wherein the drape has one portion of such a fastening system so that the pouch is movable upon the drape itself, thereby enabling a surgical team the flexibility of locating and securing the pouch in an optimal position for the procedure to be performed. To ensure proper fluid runoff into the pouch, a portion of the drape such as a fenestration reinforcement material typically provided as part of the drape may be caused to overlie an interface between the drape and that location where the pouch is attached so that the fenestration lies partially within the open top of the pouch thereby directing fluid flow into the pouch.

If the pouch were made removable, once the procedure had been completed the pouch could be removed from the drape and disposed of. At that time, a subsequent pouch may be attached if desired. This flexibility enables the pouch to be used as a waste container for waste generated in the procedure or as a disposal system for the catheter once the catheter has served its purpose. Should the pouch be used as a waste container, a liquid tight closure 42 may be provided at the otherwise open top 24. This closure 42 may comprise a channel structure 44 positioned so as to engage a mating rib 48 thereby sealing the pouch 12 when the rib is engaged with the channel. Alternatively if liquid tight sealing is not necessary, a wire tie 50 may be provided to close the pouch as shown in FIG. 2.

In use, the pouch 12 would be sterile. This could be accomplished by sterilizing the pouch alone, or in combination with the drape. When the catheter is fed into the pouch, it would snake up and around the fold because the pouch would take on a “u-shape” due to its being doubled over or loosely folded in half. The catheter may be pulled from the pouch as it is fed into the patient's artery. This eliminates the need for the catheter to be entirely removed from the pouch prior to performing the procedure. Feeding the catheter directly from the pouch into the patient's artery serves to minimize the potential for the catheter contacting a non-sterile surface. During the procedure wipes, gauze, and/or disposable instruments may be placed in the pouch for subsequent disposal. After the procedure is completed, the catheter may be removed and redirected back into the pouch. This arrangement should minimize the occurrence of blood and/or other fluids contaminating the surgical arena or contacting members of the surgical team.

It is contemplated that the surgical drape system constructed in accordance with the present invention will be tailored and adjusted by those of ordinary skill in the art to accommodate various levels of performance demand imparted during actual use. Accordingly, while this invention has been described by reference to certain specific embodiments and examples, it will be understood that this invention is capable of further modifications. This application is, therefore, intended to cover any variations, uses or adaptations of the invention following the general principles thereof, and including such departures from the present disclosure as come within known or customary practice in the art to which this invention pertains and fall within the limits of the appended claims.

1. A catheter pouch comprising:
a first sheet superimposed and sealed to a second sheet so as to create an envelope having an open top, sealed sides, and a sealed bottom; the open top fastened proximal to the sealed bottom so that a first portion of a first surface is juxtaposed with a second portion of the first surface thereby overlapping the envelope so as to form a folded pouch adapted to contain a catheter therein; and
a fastener affixed to the second sheet for securing the pouch to a surface.

2. The pouch of claim 1 wherein the sealed portions of the envelope are liquid tight.

3. The pouch of claim 1 wherein the open top comprises a closure.

4. The pouch of claim 3 wherein the closure is liquid tight.

5. The pouch of claim 1 wherein the fastener is removably securable to the surface.

6. The pouch of claim 5 wherein the fastener comprises a hook and loop fastener.

7. The pouch of claim 5 wherein the fastener comprises tape.

8. The pouch of claim 1 wherein the surface comprises a surgical drape.

9. The pouch of claim 1 wherein the pouch comprises a nonwoven material.

10. The pouch of claim 1 wherein the fastener comprises a hook and loop fastener.

11. A surgical drape system for use with a catheterization procedure comprising:
a drape having a main sheet for covering at least a substantial portion of a patient and a fenestration through the main sheet for access to a catheterization site; and
a pouch located proximal to the catheterization site for containing a catheter for use in the procedure, the pouch comprising a first surface and a second surface secured to one another so as to form a top edge and a bottom edge joined by two opposing side edges, the side edges and bottom edge being sealed closed, the top edge being fastened proximal to the bottom edge so that a first portion of the first surface is juxtaposed with a second portion of the first surface thereby overlapping the pouch into a folded orientation.

12. The system of claim 11 wherein the first and second portions of the first surface are removably fastenable to one another to enable placement and control of medical lines and tubes between the first and second portions.

13. The system of claim 11 wherein the pouch is removably fastenable to the surgical drape

14. The system of claim 12 wherein the pouch is adapted to collect fluid run-off.

15. The system of claim 11 wherein the pouch is separable from the drape and adapted to collect and seal within, for disposal; the catheter, medical waste, and fluid run-off resulting from the catheterization procedure while enabling the drape to remain in place for performance of subsequent procedures.

16. A method of manufacturing a pouch comprising:
providing a sheet material having a plurality of edges, two of the edges comprising a first and a second edge disposed opposite one another so as to define a length from the first edge to the second edge;
overlapping the sheet material so that the first and second edges are placed proximal to one another;
sealing the overlapping sheet material to itself to define a third edge and a fourth edge disposed opposite one another so as to create an envelope having an open top defined by the first and second edges, sealed sides defined by the third and fourth edges, and a sealed bottom;
overlapping the envelope so that the sealed bottom is placed proximal to the open top;
fastening the sealed bottom proximal to the open top thereby forming a pouch folded upon itself
